(19) United States
(12) Patent Application Publication
(10) Pub. No.: US 2023/0281806 A1
(43) Pub. Date: Sep. 7, 2023

(54) MICROBUBBLE COUNTING METHOD FOR PATENT FORAMEN OVALE (PFO) BASED ON DEEP LEARNING

(71) Applicant: Henan University of Science and Technology, Luoyang (CN)

(72) Inventors: Mingchuan ZHANG, Luoyang (CN); Mengjie GU, Luoyang (CN); Lin WANG, Luoyang (CN); Qingtao WU, Luoyang (CN); Junlong ZHU, Luoyang (CN); Zhihang JI, Luoyang (CN)

(73) Assignee: Henan University of Science and Technology, Luoyang (CN)

(21) Appl. No.: 18/115,726

(22) Filed: Feb. 28, 2023

(30) Foreign Application Priority Data
Mar. 7, 2022 (CN) 202210224137.X

(51) Int. Cl. G06T 7/00; G06T 5/00; G06T 5/50; G06T 7/11; G06V 10/25; G06V 10/44; G06V 10/764

(52) U.S. Cl. CPC G06T 7/0012; G06T 5/002; G06T 5/50; G06T 7/11; G06V 10/25; G06V 10/44; G06V 10/764; G06T 2207/10132; G06T 2207/20084; G06T 2207/20221; G06T 2207/30048; G06T 2207/30242

(57) ABSTRACT

A microbubble counting method for patent foramen ovale (PFO) based on deep learning is provided. The method includes: segmenting a target area of a left heart in an ultrasonic image; and generating a corresponding density map for a segmented target image using a convolutional neural network (CNN), and calculating a total number of the microbubbles in the segmented area by integration and summation. The method has the following beneficial effects: target segmentation is performed on the left atrium and left ventricular area of the heart using the neural network, and effective segmentation of the target area of the left heart is the key of obtaining parameters such as a size and form of the target area. The target area is quantitatively analyzed according to a segmentation result, and the number of the microbubbles in the target area is counted.

### CROSS REFERENCE TO RELATED APPLICATION

This patent application claims the benefit and priority of Chinese Patent Application No. 202210224137.X, filed with the China National Intellectual Property Administration on Mar. 7, 2022, the disclosure of which is incorporated by reference herein in its entirety as part of the present application.

### TECHNICAL FIELD

The present disclosure relates to the technical field of image segmentation, and in particular, to a microbubble counting method for patent foramen ovale (PFO) based on deep learning.

### BACKGROUND

The foramen ovale is a gap presented in the septum between the left and right atria. The foramen ovale usually closes gradually after birth, and the atria cannot be connected. The case that the foramen ovale is not closed after 3 years of age is known as PFO. The PFO is one of the most common congenital heart diseases in adults. The PFO is present in 40-50% of unexplained strokes. If the level of PFO is determined at an early stage and the foramen ovale is blocked through intervention or surgery, serious consequences such as cerebral stroke caused by expansion of the foramen ovale can be prevented. Therefore, it is significant to accurately identify the level of the foramen ovale gap for the diagnosis of the PFO.

In clinical practice, the classification of PFO is time-consuming and labor-intensive, moreover, there are errors among classification results made by different doctors or the same doctor at different times, due to different diagnosis experience and environment. Recently, the rise and development of big data and artificial intelligence theories provide new research ideas for modern auxiliary diagnosis and treatment. The intelligent classification of PFO provides a unified classification standard for this disease, and helps to improve the decision-making and sensitivity of doctor during diagnosis and improve the intelligence and standardization of the auxiliary diagnosis and treatment system. Therefore, clinical medicine can adapt to the current diagnosis and treatment needs under the background of “Internet”, which is the trend of medical development.

At present, scholars around the world have provided a variety of methods for intelligent diagnosis of various diseases, but the research on the intelligent classification of PFO is still in the preliminary stage. Echocardiography has become the preferred means for heart visualization because of the low cost, no known risk and contrast-enhanced ultrasound technology. Recently, a lot of intelligent classification methods for PFO based on the echocardiography have been proposed. Although they have realized the intelligent classification of the PFO, there are still many shortcomings. For example, although the classification method based on the intensity of gray value of the lesion area has achieved intelligent classification of the disease to a certain extent, its recognition rate is still low and its actual implementation is not strictly in accordance with the criteria of medical diagnosis for PFO, and thus such method still has some defects.

The primary problem to be solved for intelligent classification is to promote the segmentation accuracy of lesion regions in ultrasonic images and conduct quantitative analysis according to the clinical classification standard based on transthoracic echocardiography combined with right heart contrast echocardiography.

### SUMMARY

An objective of the present disclosure is to provide a microbubble counting method for PFO based on deep learning, so as to solve the problems of low segmentation precision of the ultrasonic image and inaccurate counting of the microbubbles in classification of the PFO in the prior art.

To achieve the above objective, the present disclosure provides the following solutions:

The present disclosure provides microbubble counting method for intelligent classification of PFO based on deep learning, including:
step 1, segmenting a target area of a left heart in an ultrasonic image; and
step 2, generating a corresponding density map for a target image of a segmented area by using a convolutional neural network (CNN), and calculating a total number of microbubbles in the segmented area by integration and summation.

Optionally, the segmenting a target area of a left heart in an ultrasonic image in step 1 are as follows:
encoding step, configured for: inputting the ultrasonic image, and performing feature extraction through a double-layer convolution operation to obtain effective features for subsequent use; reducing dimensions of the features through a pooling operation, so as to remove redundant information, simplify complexity of a network, and reduce an amount of calculation; and subjecting the features to dimension reduction for four times to extract main feature information of the ultrasonic image;
decoding step, configured for: performing a deconvolution operation on the features subjected to the dimension reduction, to restore the dimensions of the features to original resolution, and synchronously introducing features rich in shallow information through a skip-connection operation, to generate a segmented binary image; and outputting results, which specifically includes performing classification using a 1*1 convolutional layer, and outputting foreground and background layer;
post-processing step, configured for: performing, by a filter, a smoothing processing on the binary image generated through segmentation of the target area, to obtain a binary image with smooth edges; and superimposing the binary image with the original image to realize the segmentation of the left heart target area.

Optionally, the calculating a total number of microbubbles in the segmented area in step 2 are as follows: inputting the target image into ASNet and DANet, where one branch of the ASNet is a density estimation branch to generate an intermediate density map, and the other branch is an attention scaling branch to generate a scaling factor; and the DANet provides the ASNet with attention masks for relevant areas with different density levels, the ASNet multiplies the scaling factor, the intermediate density map, and the attention mask to obtain an output density map, and adds all of output density maps to obtain a final density map, and the number of the microbubbles in the target area of the left heart is obtained by integrating the density maps.

To achieve the above objective, the present disclosure further provides the following solutions:

A microbubble counting method for PFO based on deep learning includes:
obtaining a to-be-processed echocardiography video;
inputting the to-be-processed echocardiography video into the left heart target area segmentation model to determine a left heart target area, where the left heart target area segmentation model includes a spatial feature extraction network, a time flow convolutional network, and a weighted fusion network; the left heart target area segmentation model is obtained after being trained with a first training sample set; and each training sample in the first training sample set includes an echocardiography video sample and a target position of a left heart cavity in each video image frame of the echocardiography video sample;
determining to-be-counted target areas according to the left heart target area and the to-be-processed echocardiography video;
inputting the to-be-counted target areas into a counting density map model to obtain to-be-counted density maps, where the counting density map model is obtained by training a DANet network and an ASNet network with a second training sample set; and each training sample in the second training sample set includes a left heart target area sample and a microbubble density map corresponding to the left heart target area sample;
inputting the to-be-counted target areas into an attention Transformer model to obtain a papillary muscle position set, where the attention Transformer model is obtained by training a Transformer network with a third training sample set; and each training sample in the third training sample set includes a left heart target area sample and a position of papillary muscle corresponding to the left heart target area sample; and
calculating a number of microbubbles in the left heart target area corresponding to the to-be-processed echocardiography video according to the to-be-counted density maps and the papillary muscle position set.

Optionally, the spatial feature extraction network is configured for performing feature extraction on a labeled video image frame to obtain a corresponding target position feature map. The labeled video image frame may be any video image frame in the to-be-processed echocardiography video.

The time flow convolutional network is configured to extract an image pixel displacement vector with the labeled video image frame as a key frame by using an optical flow method according to the to-be-processed echocardiography video, so as to obtain a key frame target position feature map.

The weighted fusion network is configured for performing weighted fusion on multiple target position feature maps and the key frame target position feature map corresponding to each of the target position feature maps to obtain the left heart target area of.

Optionally, the spatial feature extraction network is a U-Net network. The U-Net network includes an encoding module, a decoding module, and a classification module.

An input terminal of the encoding module is configured to input the labeled video image frame.

The encoding module includes four convolutional dimension reduction submodules connected in sequence. Each of the convolutional dimension reduction submodules includes a double-layer convolution unit and a pooling dimension reduction unit connected in sequence.

The decoding module includes an input terminal connected with an output terminal of the encoding module and an output terminal configured to output a left heart target feature map corresponding to the labeled video image frame.

The decoding module includes four up-sampling modules connected in sequence. The up-sampling modules are in one-to-one correspondence with the convolutional dimension reduction submodules. Each of the up-sampling modules includes a deconvolution unit and a splicing unit. The splicing unit is configured to splice features output by the deconvolution unit with features output by a convolutional dimension reduction submodule corresponding to the deconvolution unit.

The classification module is configured for performing binary classification on the received left heart target feature map corresponding to the labeled video image frame to output the target position feature map.

Optionally, the determining to-be-counted target areas according to the left heart target area and the to-be-processed echocardiography video specifically includes:
overlapping and comparing the left heart target area with each frame of video frame sequence of the to-be-processed echocardiography video to obtain each target area frame, where multiple target area frames constitutes the to-be-counted target areas.

Optionally, the Transformer network includes a convolutional neural subnetwork, a Transformer encoder, and a Transformer decoder.

The convolutional neural subnetwork is configured for performing feature extraction on the to-be-counted density maps to obtain a density feature map.

An input terminal of the Transformer encoder is connected with an output terminal of the convolutional neural subnetwork, and the Transformer encoder is configured to encode papillary muscle in the density feature map to determine a corresponding papillary muscle number.

An input terminal of the Transformer decoder is connected with an output terminal of the Transformer encoder, and the Transformer decoder is configured to perform associated query of the papillary muscle based on a query-key mechanism to determine the position of the papillary muscle.

Optionally, the calculating a number of microbubbles in the left heart target area corresponding to the to-be-processed echocardiography video according to the to-be-counted density maps and the papillary muscle position set specifically includes:
removing corresponding point positions in the to-be-counted density maps according to the papillary muscle position set to obtain a final density map; and
performing density integration on the final density map to determine the number of the microbubbles in the left heart target area corresponding to the to-be-processed echocardiography video.

Optionally, the method for counting microbubbles in PFO based on deep learning further includes:
determining a microbubble number level of the to-be-processed echocardiography video, based on a preset standard level of microbubble number, according to the number of the microbubbles in the left heart target area corresponding to the to-be-processed echocardiography video.

According to specific embodiments provided by the present disclosure, the present disclosure discloses the following technical effects:

Target segmentation is performed on a left atrium and left ventricular area of the heart using the neural network, and effective segmentation of the target area of the left heart is the key of obtaining parameters such as a size and form of the target area. The target area is quantitatively analyzed according to a segmentation result, and the number of the microbubbles in the target area is counted, which can effectively realize intelligence and standardization of an auxiliary diagnosis process and improve working efficiency.

### BRIEF DESCRIPTION OF THE DRAWINGS

To describe the technical solutions in the embodiments of the present disclosure or in the prior art more clearly, the accompanying drawings required for the embodiments are briefly described below. Apparently, the accompanying drawings in the following description show merely some embodiments of the present disclosure, and those of ordinary skill in the art may still derive other accompanying drawings from these accompanying drawings without creative efforts.

FIG. 1 is an overall frame diagram of a microbubble counting method for PFO based on deep learning of the present disclosure;

FIG. 2 is a frame diagram of segmentation of a target area of a left heart based on an ultrasonic sectional image of the present disclosure;

FIG. 3 is a flow diagram of a microbubble counting method for PFO based on deep learning in Embodiment II the present disclosure;

FIG. 4 is a frame diagram of a left heart target area segmentation model in Embodiment II of the present disclosure;

FIG. 5 is a frame diagram of a counting density map model in Embodiment II of the present disclosure; and

FIG. 6 is a frame diagram of an attention Transformer model in Embodiment II of the present disclosure.

### DETAILED DESCRIPTION OF THE EMBODIMENTS

The technical solutions of the embodiments of the present disclosure are clearly and completely described below with reference to the accompanying drawings. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

To make the above-mentioned objective, features, and advantages of the present disclosure clearer and more comprehensible, the present disclosure will be further described in detail below in conjunction with the accompanying drawings and specific embodiments.

### Embodiment I

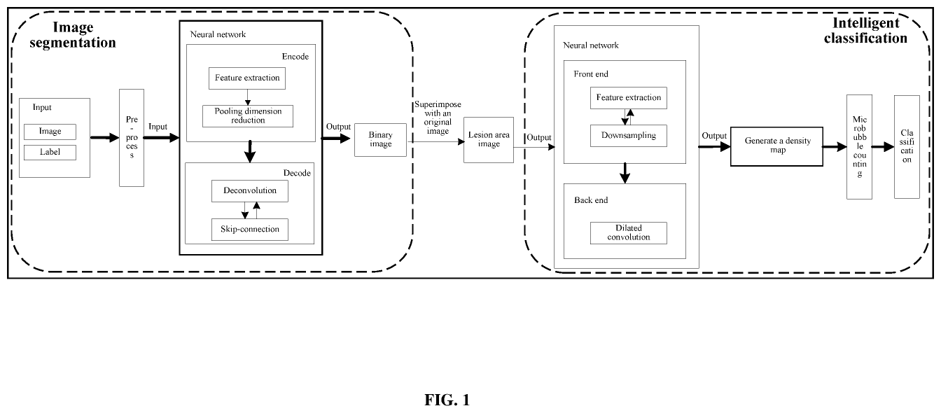

In the present disclosure, transthoracic echocardiography combined with right heart contrast echocardiography is a commonly used method for diagnosing PFO in clinical practice. The size and shape of the target area of the left heart are important parameters to determine whether the heart is normal. According to the right-to-left shunting characteristics of the heart with PFO, normal saline with bubbles is injected into the human body. The disease grade of PFO can be determined by observing the number of microbubbles in the left ventricular area after several cardiac cycles. The present disclosure provides a microbubble counting method for PFO based on deep learning. Target segmentation is first performed on the left atrium and left ventricular area of the heart using the neural network, and effective segmentation of the target area of the left heart is the key of obtaining parameters such as a size and form of the target area. The target area is quantitatively analyzed according to a segmentation result, and the number of the microbubbles in the target area is counted. A main flow of the present disclosure is shown in FIG. 1.

The present disclosure is mainly divided into two parts. The first part is to segment a target area of a left heart in an ultrasonic image for further functional quantitative analysis and processing. The second part is to generate a corresponding density map for the clipped target image using a CNN, and calculate a total number of the microbubbles in the segmented area by integration and summation.

1. Target Segmentation

The foreground point and background point of the segmented image are firstly determined in the segmentation of the target area of the left heart. The foreground point is the point position of the left atrium and left ventricular target area, while the background point is the black area of the right atrium, right ventricle and myocardium. The original ultrasonic image and the binary image of the foreground and background points are input into the U-Net network combining low resolution and high resolution information to achieve accurate segmentation of the left ventricle in the ultrasonic sectional image. The specific work is as follows.

(1) Encoding: the ultrasonic sectional image is input, and feature extraction is performed through a double-layer convolution operation to obtain effective features for subsequent use. Dimensions of the features are reduced through a pooling operation, so as to remove redundant information, simplify complexity of a network, and reduce an amount of calculation. Finally, after four times of dimension reduction, main feature information of the image is extracted.

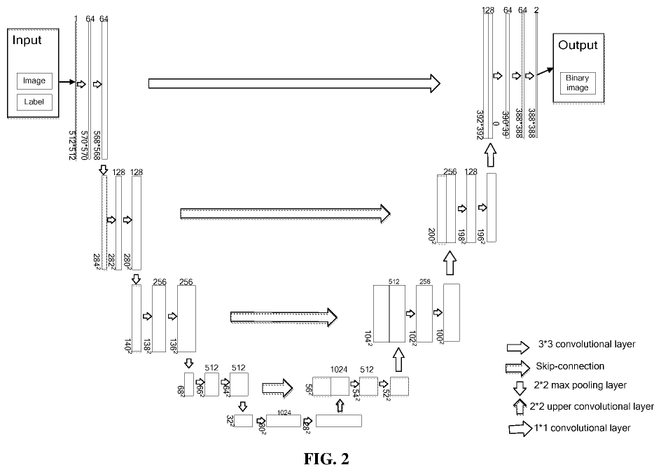

(2) Decoding: first, for the features subjected to the dimension reduction, the dimensions of the features are restored to an original resolution through a deconvolution operation. Second, during the deconvolution operation, features with rich shallow information are introduced through a skip-connection operation, so as to generate a segmentation binary image. Finally, results are output, specifically, classification is performed using a 1*1 convolutional layer, and foreground and background layers are output. A specific flow is shown in FIG. 2.

(3) Post-processing: for a problem that an edge of the binary image generated after the target area is segmented is not smooth, smoothing processing is performed using a filter to obtain a binary image with a smooth edge. The image is superimposed with the original image to segment the target area of the left heart.

2. Intelligent Classification of PFO

According to the classification criteria of PFO, the absence of microbubbles in the left heart is negative and the presence of microbubbles is positive. The positive is divided into three levels with the presence of 1-10 microbubbles as a small number level, the presence of 11-30 microbubbles as a medium number level, and the presence greater than 30 microbubbles as a large number level. Therefore, the key to realize the intelligent classification of the disease is to determine the number of microbubbles in the left ventricular area. In this method, in order to determine the number of microbubbles in the left ventricular area, the position information of microbubbles should first be determined according to the segmented area and the corresponding density map should be generated. The segmented target area and its corresponding density map should be input into the target counting neural network, so as to count the microbubbles. The specific work is as follows.

(1) Microbubble counting: the target image is input into ASNet and DANet. One branch of the ASNet is a density estimation branch to generate an intermediate density map, and the other branch is an attention scaling branch to generate a scaling factor. The DANet provides the ASNet with attention masks for relevant areas with different density levels. The ASNet multiplies the scaling factor, the intermediate density map, and the attention mask to obtain an output density map, and adds all of the output density maps to obtain a final density map. The number of the microbubbles in the target area of the left heart is obtained by integrating the density maps.

In order to solve the problems that the diagnosis of PFO is time-consuming and laborious at present in clinical practice, and there are errors in the diagnosis results of different doctors or the diagnosis results of the same doctor at different times, the present disclosure provides a microbubble counting method for PFO based on deep learning. The method includes the following steps 1 to 10.

In step 1, ultrasonic sectional image data is obtained.

In step 2, a position of the left endocardium is manually marked in the obtained ultrasonic image.

In step 3, the marked image is pre-processed and made into a binary image as training data.

In step 4, a U-Net network is trained with the ultrasonic image and binary image. After the convolution operation, features of the image are extracted, and dimensions of the features are reduced by down-sampling. A predicted image is compared with the binary image to obtain training loss. The network model is adjusted according to the loss constantly to finally obtain the trained network model. The left heart area is segmented using the trained network model to obtain the segmented binary image.

In step 5, the edge of the black and white binary image output by the neural network is smoothed using a median filter.

In step 6, the smoothed binary image is superimposed with the original image to obtain the segmented target area.

In step 7, positions of microbubbles are manually marked in the segmented target image.

In step 8, a corresponding density map is generated as the training data according to the position information of the microbubbles.

In step 9, ASNet and DANet networks are trained with the segmented target image, and the corresponding microbubble density map of the target area is generated using the trained model.

In step 10, a number of microbubbles is calculated by integrating and summing the microbubble density map generated.

### Embodiment II

Due to its safety, non-invasiveness and high detection rate, transthoracic right heart contrast echocardiography is a commonly used method in clinical diagnosis of PFO. The size and shape of the left ventricular area are important parameters to determine whether the heart is normal. According to the right-to-left shunting characteristics of the heart with PFO, normal saline with bubbles is injected into the human body. The disease grade of PFO can be determined by observing the number of microbubbles in the left ventricular area after several cardiac cycles.

Based on this principle, the present disclosure provides a microbubble counting method for PFO based on deep learning. First, segmentation is performed on the left atrium and left ventricular area of the heart using the neural network, and effective segmentation of the left ventricular area is the key of obtaining parameters such as a size and form of the left heart cavity. Second, the left heart cavity area is quantitatively analyzed according to a segmentation result. A corresponding density map is generated for the segmented left heart cavity using a CNN. In addition, the papillary muscle in the heart cavity is tracked using a Transformer architecture. Then, the density map is modified according to tracking results, and the papillary muscle is removed from the corresponding position in the density map. Finally, the total number of microbubbles in the segmented area is calculated by integration and summation, and the classification is performed to realize intelligent auxiliary diagnosis of PFO.

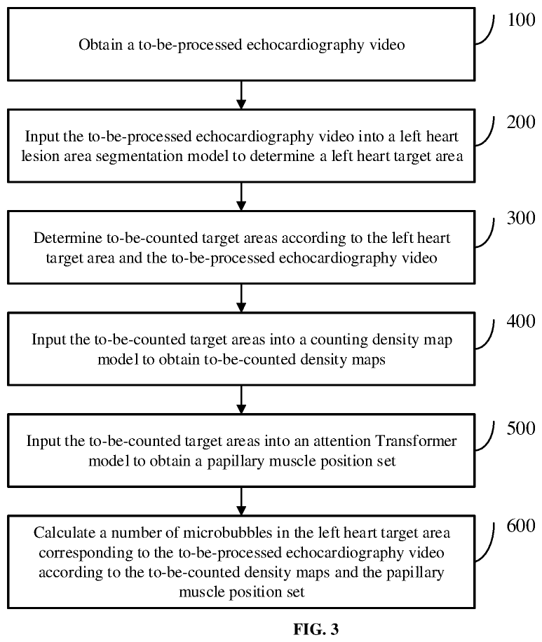

Specifically, as shown in FIG. 3, the microbubble counting method for PFO based on deep learning includes the following steps 100 to 600.

In step 100, a to-be-processed echocardiography video is obtained.

In step 200, the to-be-processed echocardiography video is input into a left heart target area segmentation model to determine a left heart target area. The left heart target area segmentation model is a double-flow network. The left heart target area segmentation model includes a spatial feature extraction network, a time flow convolutional network, and a weighted fusion network. Both an output terminal of the spatial feature extraction network and an output terminal of the time flow convolutional network are connected with an input terminal of the weighted fusion network. The left heart target area segmentation model is obtained by training with a first training sample set. Each training sample in the first training sample set includes an echocardiography video sample and a target position of a left heart cavity on each video image frame of the echocardiography video sample. The target position of the left heart cavity on each video image frame of the echocardiography video sample can be manually marked by doctors.

Specifically, in order to eliminate the interference of the right atrium on subsequent data processing, the left atrium and the right atrium in the to-be-processed echocardiography video are segmented. In the actual video image, the foreground point and background point of the segmented actual video image are determined. The foreground point is the point position of the left atrium and left ventricle, while the background point is the black area of the right atrium, right ventricle and myocardium.

In the transthoracic right heart contrast echocardiography, the microbubbles are in a moving state, and the movement of the microbubbles is irregular. The movement of the myocardium has a pattern and can be recognized. In order to further improve the accuracy of the left heart target area segmentation model for image segmentation, the steps of recognition and extraction of a myocardial motion relationship in the echocardiography video sample are added to the left heart target area segmentation model, namely, the left heart target area segmentation model includes the spatial feature extraction network and the time flow convolutional network.

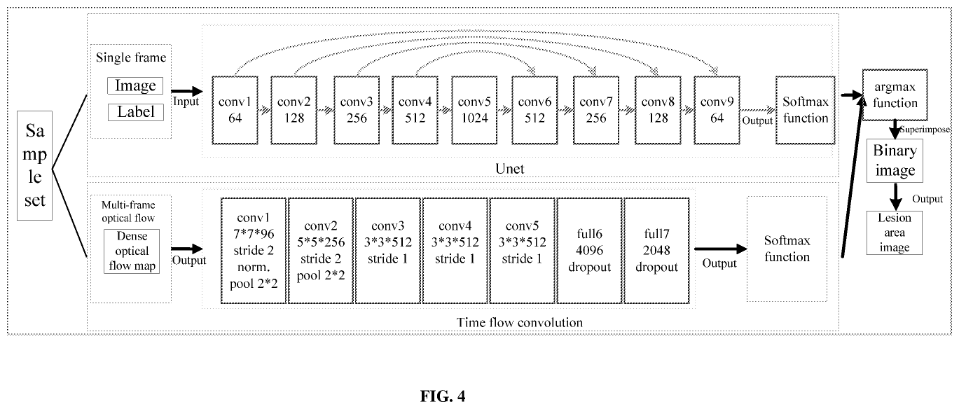

In the left heart target area segmentation model, as shown in FIG. 4, the spatial feature extraction network is configured for feature extraction of a labeled video image frame to obtain a corresponding target position feature map. The labeled video image frame is any video image frame in the to-be-processed echocardiography video. The time flow convolutional network is configured to extract an image pixel displacement vector by using an optical flow method and taking the labeled video image frame as a key frame according to the to-be-processed echocardiography video, so as to obtain a target position feature map of a key frame. The weighted fusion network is configured for weighted fusion of multiple target position feature maps and the target position feature map of a key frame corresponding to each of the target position feature maps to obtain the target area of the left heart.

Specifically, the spatial feature extraction network is a U-Net network. The U-Net network includes an encoding module, a decoding module, and a classification module. An input terminal of the encoding module is configured to input the labeled video image frame. The encoding module includes four convolutional dimension reduction submodules connected in sequence, which correspond to 64-dimensional Convolution 1, 128-dimensional Convolution 2, 256-dimensional Convolution 3, and 512-dimensional Convolution 4 in FIG. 4. Each of the convolutional dimension reduction submodules includes a double-layer convolution unit and a pooling dimension reduction unit connected in sequence. Features of each video frame in the input echocardiography video are extracted through a double-layer convolution unit to obtain effective features for subsequent use. Dimensions of the features are reduced through a pooling operation, so as to remove redundant information, simplify complexity of a network, and reduce an amount of calculation. Finally, after four times of dimension reduction, main feature information of the image is extracted.

The decoding module includes an input terminal connected with an output terminal of the encoding module and an output terminal configured to output a left heart target feature map corresponding to the labeled video image frame. The decoding module includes four up-sampling modules connected in sequence, which correspond to 512-dimensional Convolution 6, 256-dimensional Convolution 7, 128-dimensional Convolution 8, and 64-dimensional Convolution 9 in FIG. 4. In addition, Convolution 6 and Convolution 4 are connected by Convolution 5, which has 1,024 dimensions. The up-sampling modules are in one-to-one correspondence with the convolutional dimension reduction submodules. Each of the up-sampling modules includes a deconvolution unit and a splicing unit. The splicing unit is configured to splice features output by the deconvolution unit with features output by a convolutional dimension reduction submodule corresponding to the deconvolution unit. Specifically, in the decoding stage, for the features subjected to the dimension reduction, the dimensions of the features are restored to an original resolution through a deconvolution operation. During the deconvolution operation, features with rich shallow information are introduced through a skip-connection operation, so as to generate a segmentation binary image.

The classification module is configured for binary classification of the received left heart target feature map corresponding to the labeled video image frame to output the target position feature map. Specifically, a binary probability map is generated by a softmax function after decoding.

The time flow convolutional network includes a first convolutional layer, a second convolutional layer, a third convolutional layer, a fourth convolutional layer, a fifth convolutional layer, a sixth linear layer, and a seventh linear layer connected in sequence. The time flow convolutional network firstly extracts a dense optical flow sequence from the to-be-processed echocardiography video by the optical flow method. The dense optical flow sequence includes multiple dense optical flow maps. Then, L+1 frames (which can be set according to the actual needs, generally speaking, more intercepted frames indicates more accurate results) are intercepted later by taking the labeled video image frame as the key frame to obtain L optical flow maps. The above L optical flow maps are convolved to make the network learn the motion information of the heart cavity. Finally, the softmax function is used to output a binary probability map, namely the target position feature map of a key frame.

The dense optical flow sequence is extracted from the to-be-processed echocardiography video by the optical flow method, specifically as follows. The optical flow refers to the movement of target pixels caused by the movement of microbubbles in two consecutive video frame sequences, representing the displacement of a point from the first frame to the second frame. The output of the optical flow method is an estimated correlation of the speed of each pixel in two video frame sequences, or the displacement vector of each pixel in one image, indicating the relative position of that pixel in the other image.

For example, the pixel I(x, y, t) in the first frame represents the value of pixel I(x, y) at a time t. After the dt time, the pixel moves (dx, dy) in the next frame. Since the pixels are the same and the intensity remains unchanged, it can be expressed as: I(x, y, t)=I(x+dx, y+dy, t+dt), which is the motion relationship sequence of the second frame. The position of the heart cavity in the second frame can be obtained according to the motion relationship sequence, which is performed in sequence orderly, and the myocardial motion relationship sequence can be obtained until the last frame image, namely the dense optical flow sequence mentioned above.

The weighted fusion network specifically makes the weighted average of the binary probabilities output by the spatial feature extraction network and the time flow convolutional network to obtain a final predicted probability. Then, through an argmax operation, a maximum of binary classification after the weighted average is selected. The foreground point and background point of the image are determined to output a black and white binary image. The black and white binary image is superimposed with the original image to obtain a segmented target area of the left heart.

In conclusion, based on the video frame sequence, the single video frame and the position of the target of left heart corresponding to the single video frame are input into the U-Net network combining low resolution and high resolution information to obtain spatial feature information of the single video frame. Due to the time sequence between multi-frame video frames and the temporal feature information in terms of spatial feature information, the multi-frame video frames are input into the time flow convolutional network to extract the myocardial motion relationship based on the optical flow method. The target position feature map of a key frame is further determined. Finally, weighted fusion is performed to obtain the final target area of the left heart.

In order to further obtain a more accurate target area of the left heart, for a problem that an edge of the binary image (the target area of the left heart) generated after the left heart cavity segmentation is not smooth, smoothing processing is performed using a filter to obtain a binary image with a smooth edge.

In step 300, to-be-counted target areas are determined according to the target area of the left heart and the to-be-processed echocardiography video.

Step 300 specifically includes: overlapping and comparing the target area of the left heart with each frame of video frame sequence of the to-be-processed echocardiography video to obtain each frame of target area. Multiple frames of the target area constitutes the to-be-counted target areas. That is, the obtained black and white binary image is superimposed with the to-be-processed echocardiography video before segmentation to achieve accurate segmentation of the left ventricle based on echocardiography.

In step 400, the to-be-counted target areas are input into a counting density map model to obtain to-be-counted density maps. The counting density map model is obtained by training a DANet network and an ASNet network with a second training sample set. Each training sample in the second training sample set includes a left heart target area sample and a microbubble density map corresponding to the left heart target area sample.

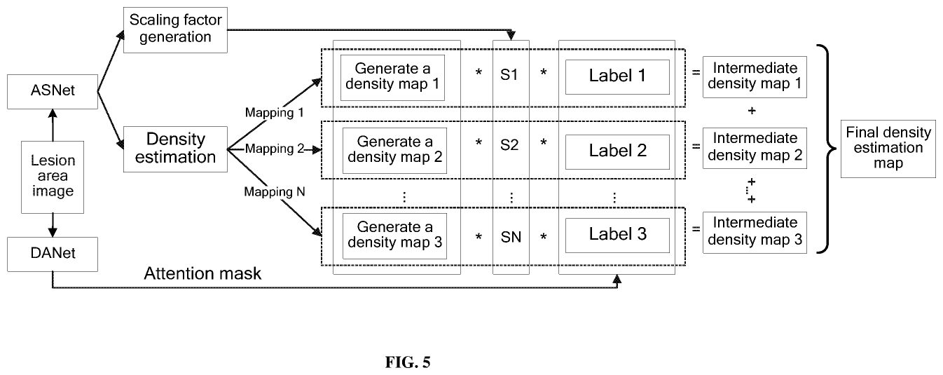

Specifically, as shown in FIG. 5, the left heart cavity images (to-be-counted target areas) are input into the ASNet network and the DANet network. The ASNet network includes two branch networks. One branch is a density estimation branch to generate an intermediate density map, and the other branch is an attention scaling branch to generate a scaling factor. The DANet network provides the ASNet network with attention masks for relevant areas with different density levels. The ASNet network multiplies the scaling factor, the intermediate density map, and the attention mask to obtain an output density map, and adds all of the output density maps to obtain final density maps, namely, the to-be-counted density maps.

In step 500, the to-be-counted target areas are input into an attention Transformer model to obtain a papillary muscle position set. The attention Transformer model is obtained by training a Transformer network with a third training sample set. Each training sample in the third training sample set includes a left heart target area sample and a position of papillary muscle corresponding to the left heart target area sample.

It is an important problem in the counting of microbubbles in PFO to reflect the real-time movement of microbubbles and papillary muscle. Due to the rapid movement of microbubbles, overlapping often occurs in the transthoracic right heart contrast echocardiography. In addition, the papillary muscle in the heart cavity has similar grayscale features with microbubbles during movement, which is easy to be mistaken for microbubbles in the counting process of density map. In order to solve the phenomenon of suspected microbubbles in papillary muscle imaging and accurately count the microbubbles, the Transformer architecture based on encoder and decoder is adopted in the present embodiment to establish the corresponding relationship between cross-frame pixels.

The Transformer Network includes a convolutional neural subnetwork, a Transformer encoder, and a Transformer decoder. The papillary muscle features from the convolutional network are encoded by the Transformer encoder, and the query vector is decoded into the papillary muscle enclosure and corresponding identity (ID) by the Transformer decoder. The tracking query is used for data correlation between frames.

Specifically, the convolutional neural subnetwork is configured for feature extraction of the to-be-counted target maps to obtain a target feature map. An input terminal of the Transformer encoder is connected with an output terminal of the convolutional neural subnetwork, and the Transformer encoder is configured to encode papillary muscle in the target feature map to determine a corresponding papillary muscle number. An input terminal of the Transformer decoder is connected with an output terminal of the Transformer encoder, and the Transformer decoder is configured to perform associated query of the papillary muscle based on a query-key mechanism to determine the position of the papillary muscle.

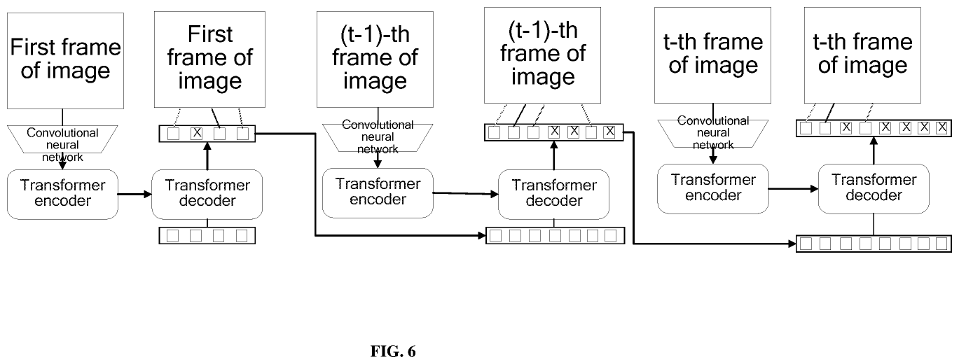

In practical applications, as shown in FIG. 6, the first frame of to-be-counted target areas (in form of image) are inputted to the CNN to extract features of the input density image, and the extracted features are input into the Transformer encoder, which encodes the papillary muscles in the image, namely, giving each papillary muscle a number. Since it is the first frame, there is no need to perform a target association operation with the previous frame. Therefore, the step of using the query-key mechanism to carry out target association query is omitted in the decoder, and the corresponding position of the papillary muscle is directly circled in the first frame of to-be-counted target areas and recorded in the tracker. For the second frame of to-be-counted target areas, the CNN is also used first for feature extraction and the encoder is used for encoding operation of the papillary muscle. During decoding, the query-key mechanism is used to realize the associated query with the target of the previous frame to realize the tracking function. The corresponding position of the papillary muscle is circled in the second frame of to-be-counted target areas and recorded in the tracker. The operation of each subsequent frame of to-be-counted target areas can be performed in a similar fashion to obtain the papillary muscle position set.

In step 600, a number of microbubbles in the target area of the left heart corresponding to the to-be-processed echocardiography video is calculated according to the to-be-counted density maps and the papillary muscle position set.

Step 600 specifically includes the following steps.

1) Corresponding point positions in the to-be-counted density maps are removed according to the papillary muscle position set to obtain a final density map.

2) Density integration is performed on the final density map to determine the number of the microbubbles in the target area of the left heart corresponding to the to-be-processed echocardiography video.

The microbubble counting method for PFO based on deep learning in the present embodiment further includes: based on a preset microbubble number standard level, determining a microbubble number level of the to-be-processed echocardiography video according to the number of the microbubbles in the target area of the left heart corresponding to the to-be-processed echocardiography video.

The preset microbubble number standard level is a clinical classification standard for the PFO.

In conclusion, the present embodiment is mainly divided into two parts. The first part is to dynamically segment the left ventricular area of echocardiography by using the optical flow method combined with the U-Net network, so as to facilitate further functional quantitative analysis and processing. In the second part, the corresponding density map is generated using the CNN for the segmented left heart cavity, and the Transformer architecture is used to track the papillary muscle in the heart cavity. Then, according to the tracking results, the microbubble error counting is determined at the corresponding position in the density map, and the density map is modified. Finally, the total number of microbubbles in the segmented area is calculated by integration and summation, and the classification is performed to realize intelligent auxiliary diagnosis of the PFO.

Compared with Embodiment I, which directly integrates the density map to calculate the number of microbubbles in the left ventricular area, the present embodiment can achieve more accurate counting. The to-be-processed echocardiography video in the present embodiment includes multi-frame video frames. When the number of video frames is 1, a single echocardiogram is processed. In practical applications, faced with a large amount of data, the technical solution in Embodiment I is generally adopted in order to improve the efficiency of microbubble counting. In the case of a small amount of data, the technical solution in the present embodiment is generally adopted in order to improve the accuracy of microbubble counting.

In order to solve the problems that the microbubble counting in the PFO is time-consuming and laborious at present in clinical practice, and there are errors in the diagnosis results of different doctors or the diagnosis results of the same doctor at different times, in a specific embodiment, the classification method for PFO based on echocardiography includes the following steps 1 to 11.

In step 1, echocardiography data is obtained.

In step 2, the obtained echocardiogram is split into video sequence, and the image sequence after the right heart cavity is filled with a contrast agent is selected as the subsequent study object according to the cardiac cycle.

In step 3, the selected video sequence is input into the trained left heart target area segmentation model to segment the video sequence to determine a finally-predicted left heart target area. The left heart target area segmentation model is a double-flow network. The key focus location features are extracted through the spatial feature extraction network. The dense optical flow sequences in the video sequences are extracted based on the optical flow method through the time flow convolution network to obtain the myocardial motion relationship sequences. Then, the key focus location features and the myocardial motion relationship sequences are fused to obtain the accurate left heart focus area. In the process of training the left heart focus area segmentation model, it is necessary to constantly adjust the network model according to the loss function to finally obtain the trained left heart focus area segmentation model.

In step 4, the finally predicted relationship diagram is made into a binary image.

In step 5, edge smoothing processing is performed on the black and white binary image output in step 4 using a median filter.

In step 6, the smoothed binary image is superimposed with the original image (the echocardiogram obtained in step 1) to obtain the segmented left heart cavity.

In step 7, the segmented left heart cavity image is input into the trained ASNet and DANet networks to obtain the microbubble density map corresponding to the left ventricular area. The training process of the ASNet and DANet networks includes: manually marking the position of microbubbles in the left ventricular sample image, generating the corresponding density map as training data according to the position information of the microbubbles, and training the ASNet and DANet networks with the training data to obtain an optimal network model.

In step 8, the myocardial tissue in the segmented left heart target area is tracked using Transformer and the position of the papillary muscle is marked in the corresponding density map.

In step 9, the corresponding points are removed from the density map according to the positions marked in step 8.

In step 10, the finally generated microbubble density map is integrated and summed to calculate the number of microbubbles.

In step 11, the PFO is classified according to the diagnostic criteria.

Focusing on the difficult problem of classification of PFO based on transthoracic right heart contrast echocardiography, the present disclosure actively explores the classification method for PFO based on deep learning technology, studies the cooperative expression mechanism of multi-layer feature semantics based on the CNN, develops an efficient classification model of ultrasonic images guided by prior knowledge, and establishes a prototype verification system with independent intellectual property rights. The intelligence and standardization of an auxiliary diagnosis process is realized and the efficiency of diagnosis is improved. The research of the present disclosure provides key theoretical and technical support for the intelligent development of the diagnosis of PFO, and also provides important reference for the diagnosis methods related to the disease.

Based on the classification concept of traditional medical treatment, the present disclosure introduces theoretical technologies such as big data and machine learning to perform in-depth research from two aspects of left ventricular segmentation and classification, and actively explores the intelligent method of ultrasonic accurate segmentation of the left heart cavity and benign and malignant classification of PFO. The present disclosure is expected to promote the research progress of medical intelligent assisted diagnosis theory and technology and improve the development environment of clinical diagnosis and treatment, which has important scientific significance.

Embodiments of the present specification are described in a progressive manner, each embodiment focuses on the difference from other embodiments, and the same and similar parts between the embodiments may refer to each other.

Specific examples are used herein to explain the principles and embodiments of the present disclosure. The foregoing description of the embodiments is merely intended to help understand the method of the present disclosure and its core ideas; besides, various modifications may be made by those of ordinary skill in the art to specific embodiments and the scope of application in accordance with the ideas of the present disclosure. In conclusion, the content of the present specification shall not be construed as limitations to the present disclosure.

1. A microbubble counting method for patent foramen ovale (PFO) based on deep learning, comprising:
step 1, segmenting a target area of a left heart in an ultrasonic image; and
step 2, generating a corresponding density map for a target image of a segmented area using a convolutional neural network (CNN), and calculating a total number of microbubbles in the segmented area by integration and summation.

2. The microbubble counting method for PFO based on deep learning according to claim 1, wherein the segmenting a target area of a left heart in an ultrasonic image in step 1 is as follows:
encoding step, configured for: inputting the ultrasonic image, and performing feature extraction through a double-layer convolution operation to obtain effective features for subsequent use; reducing dimensions of the features through a pooling operation, so as to remove redundant information, simplify complexity of a network, and reduce an amount of calculation; and subjecting the features to dimension reduction for four times to extract main feature information of the ultrasonic image;
decoding step, configured for: performing a deconvolution operation on the features subjected to the dimension reduction to restore the dimensions of the features to original resolution and synchronously introducing features rich in shallow information through a skip-connection operation, to generate a segmented binary image; and outputting results, which specifically includes: performing classification using a 1*1 convolutional layer, and outputting foreground and background layers; and
post-processing step, configured for: performing, by a filter, a smoothing processing on the binary image generated after segmentation of the target region, to obtain a binary image with smooth edges; and superimposing the binary image with the original image to segment the target area of the left heart.

3. The microbubble counting method for PFO based on deep learning according to claim 1, wherein the calculating a total number of microbubbles in the segmented area in step 2 are as follows: inputting the target image into ASNet and DANet, where one branch of the ASNet is a density estimation branch to generate an intermediate density map, and the other branch is an attention scaling branch to generate a scaling factor; and the DANet provides the ASNet with attention masks for relevant areas with different density levels, the ASNet multiplies the scaling factor, the intermediate density map, and the attention mask to obtain an output density map, and adds all of output density maps to obtain a final density map, and the number of the microbubbles in the target area of the left heart is obtained by integrating the density maps.

4. A microbubble counting method for PFO based on deep learning, comprising:
obtaining a to-be-processed echocardiography video;
inputting the to-be-processed echocardiography video into a left heart target area segmentation model to determine a left heart target area, wherein the left heart target area segmentation model comprises a spatial feature extraction network, a time flow convolutional network, and a weighted fusion network; the left heart target area segmentation model is obtained after being trained with a first training sample set; and each training sample in the first training sample set comprises an echocardiography video sample and a target position of a left heart cavity in each video image frame of the echocardiography video sample;
determining to-be-counted target areas according to the left heart target area and the to-be-processed echocardiography video;
inputting the to-be-counted target areas into a counting density map model to obtain to-be-counted density maps, wherein the counting density map model is obtained by training a DANet network and an ASNet network with a second training sample set; and each training sample in the second training sample set comprises a left heart target area sample and a microbubble density map corresponding to the left heart target area sample;
inputting the to-be-counted target areas into an attention Transformer model to obtain a papillary muscle position set, wherein the attention Transformer model is obtained by training a Transformer network with a third training sample set; and each training sample in the third training sample set comprises a left heart target area sample and a position of papillary muscle corresponding to the left heart target area sample; and
calculating a number of microbubbles in the left heart target area corresponding to the to-be-processed echocardiography video according to the to-be-counted density maps and the papillary muscle position set.

5. The microbubble counting method for PFO based on deep learning according to claim 4, wherein the spatial feature extraction network is configured for performing feature extraction on a labeled video image frame to obtain a corresponding target position feature map; and the labeled video image frame is any video image frame in the to-be-processed echocardiography video;
the time flow convolutional network is configured to extract an image pixel displacement vector with the labeled video image frame as a key frame by using an optical flow method according to the to-be-processed echocardiography video, so as to obtain a key frame target position feature map; and
the weighted fusion network is configured for performing weighted fusion on multiple target position feature maps and the key frame target position feature map corresponding to each of the target position feature maps to obtain the left heart target area.

6. The microbubble counting method for PFO based on deep learning according to claim 5, wherein the spatial feature extraction network is a U-Net network; and the U-Net network comprises an encoding module, a decoding module, and a classification module;
an input terminal of the encoding module is configured to input the labeled video image frame;
the encoding module comprises four convolutional dimension reduction submodules connected in sequence; and each of the convolutional dimension reduction submodules comprises a double-layer convolution unit and a pooling dimension reduction unit connected in sequence;
the decoding module comprises an input terminal connected with an output terminal of the encoding module and an output terminal configured to output a left heart target feature map corresponding to the labeled video image frame;
the decoding module comprises four up-sampling modules connected in sequence; the up-sampling modules are in one-to-one correspondence with the convolutional dimension reduction submodules; each of the up-sampling modules comprises a deconvolution unit and a splicing unit; and the splicing unit is configured to splice features output by the deconvolution unit with features output by a convolutional dimension reduction submodule corresponding to the deconvolution unit; and
the classification module is configured for performing binary classification on the received left heart target feature map corresponding to the labeled video image frame to output the target position feature map.

7. The microbubble counting method for PFO based on deep learning according to claim 4, wherein the determining to-be-counted target areas according to the left heart target area and the to-be-processed echocardiography video specifically comprises:
overlapping and comparing the left heart target area with each frame of video frame sequence of the to-be-processed echocardiography video to obtain each target area frame, wherein multiple the target area frames constitutes the to-be-counted target areas.

8. The microbubble counting method for PFO based on deep learning according to claim 4, wherein the Transformer network comprises a convolutional neural subnetwork, a Transformer encoder, and a Transformer decoder;
the convolutional neural subnetwork is configured for performing feature extraction on the to-be-counted target areas to obtain a target feature map;
an input terminal of the Transformer encoder is connected with an output terminal of the convolutional neural subnetwork, and the Transformer encoder is configured to encode papillary muscle in the target feature map to determine a corresponding papillary muscle number; and
an input terminal of the Transformer decoder is connected with an output terminal of the Transformer encoder, and the Transformer decoder is configured to perform associated query of the papillary muscle based on a query-key mechanism to determine the position of the papillary muscle.

9. The microbubble counting method for PFO based on deep learning according to claim 4, wherein the calculating a number of microbubbles in the left heart target area corresponding to the to-be-processed echocardiography video according to the to-be-counted density maps and the papillary muscle position set specifically comprises:
removing corresponding point positions in the to-be-counted density maps according to the papillary muscle position set to obtain a final density map; and
performing density integration on the final density map to determine the number of the microbubbles in the left heart target area corresponding to the to-be-processed echocardiography video.

10. The microbubble counting method for PFO based on deep learning according to claim 4, further comprising:
determining a microbubble number level of the to-be-processed echocardiography video, based on a preset microbubble number standard level, according to the number of the microbubbles in the left heart target area corresponding to the to-be-processed echocardiography video.
